(19) United States
(12) Patent Application Publication
(10) Pub. No.: US 2002/0072512 A1
(43) Pub. Date: Jun. 13, 2002

(54) Method of preventing and treating HIV-mediated central nervous system damage

(75) Inventor: Daniela Salvemini, Chesterfield, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Metaphore Pharmaceuticals, Inc

(21) Appl. No.: 09/951,855

(22) Filed: Sep. 13, 2001

Related U.S. Application Data
(60) Provisional application No. 60/254,405, filed on Dec. 8, 2000.

(51) Int. Cl. A61K 31/555

(52) U.S. Cl. 514/184; 514/185

(57) ABSTRACT

The present invention relates to methods of preventing and/or treating HIV-mediated central nervous system damage. The method comprises administering to a subject therapeutic amounts of non-proteinaceous catalysts for the dismutation of superoxide to a subject either alone or in combination with a HIV anti-viral agent. The compounds of the invention are particularly suitable for treating and/or preventing AIDS Dementia Complex.

### CROSS-REFERENCE TO RELATED APPLICATION

This application claims priority from Provisional Application Serial No. 60/254,405 filed on December 8, 2000, which is hereby incorporated by reference in its entirety.

### FIELD OF THE INVENTION

The current invention generally relates to methods of preventing and/or treating HIV-mediated central nervous system damage by administering therapeutic amounts of non-proteinaceous catalysts for the dismutation of superoxide anions to a subject. Also provided are pharmaceutical compositions comprising catalysts for the dismutation of superoxide anions.

### BACKGROUND OF THE INVENTION

HIV, in addition to infecting cells of the immune system, directly infects certain cells of the Central Nervous System (“CNS”), which comprises the brain and the spinal cord. The cells in the brain that HIV predominantly infects are those in the white matter, which include the microglia, astroglial cells, monocytes and macrophages. A direct consequence of HIV CNS infection is the development of AIDS Dementia Complex (“ADC”, also known as HIV/AIDS Encephalopathy and HIV/AIDS Related Brain Impairment) in a portion of the infected population. The classic symptoms of ADC include diminished cognitive function, impaired motor skills, and behavioral changes. It is currently estimated that approximately 20% of people with AIDS develop ADC.

The mechanism of HIV-related ADC remains to be fully elucidated. However, apoptosis of neurons and non-neuronal cells has been demonstrated in the brain of AIDS patients with dementia and is considered to be a major source of cell death (Shi et al., (1998) J. Neurovirol 4:281-290). In the apoptotic pathway, cell death is triggered by an intracellular controlled process characterized by a condensation and subsequent fragmentation of the cell nucleus during which the plasma membrane remains intact. A multitude of studies suggest that the apoptotic stimuli are likely to be soluble factors. Several candidates for the soluble factors that lead to apoptotic cell death in HIV-1 infection have been proposed, including viral proteins (e.g. gpl20, Tat; Seve et al., (1999) Arch. Biochem.Biophys. 15:165-172; Bagetta et al., (1998) Biochem. Biophys. Res. Commun. 244:819-824; Bagetta et al., (1999) Neuroscience 89:1051-1066; and Ehret et al., (1996) J. Virol. 70:6502-6507), inappropriate secretion of inflammatory cytokines by activated macrophages (i.e. tumor necrosis factor alpha, TNFa; Westendorp et al., (1995) EMBO J. 14:546-554; and Shatrov et al., Eur. Cytokine Netw 8:37-43 ) and toxins produced by opportunistic micro-organisms. Collectively, this data suggests that the mechanism(s) that lead to neuronal as well as non-neuronal apoptosis in the brain of AIDS patients in vivo, may involve the combined effect of more than one pro-apoptotic factor. However, among neurotoxic factors released by infected macrophages, clear evidence indicates that overproduction of reactive oxygen species underlies apoptotic cell death in neuroAIDS (Pace et al., (1995) Free Radic Biol Med 19:523-528; and Romero-Alvira et al., (1998) Med. Hypotheses 51:169-173).

One characteristic of patients suffering from ADC, therefore, is a large increase in the production and accumulation of free radicals, including superoxide anions (O₂⁻). Superoxide anions are normally removed in biological systems by the formation of hydrogen peroxide and oxygen in the following reaction (hereinafter referred to as dismutation):

O₂⁻+O₂⁻+2H⁺O₂+H₂O₂

This reaction is catalyzed in vivo by the ubiquitous superoxide dismutase enzyme (SOD).

Several non-proteinaceous catalysts which mimic this superoxide dismutating activity have been discovered. A particularly effective family of non-proteinaceous catalysts for the dismutation of superoxide consists of the manganese(II), manganese(III), iron(II) or iron(III) complexes of nitrogen-containing fifteen-membered macrocyclic ligands which catalyze the conversion of superoxide into oxygen and hydrogen peroxide, as described in U.S. Pat. Nos. 5,874,421 and 5,637,578, all of which are incorporated herein by reference. See also, Weiss, R.H., et al., “Manganese(II)-Based Superoxide Dismutase Mimetics: Rational Drug Design of Artificial Enzymes”, Drugs of the Future 21: 383-389 (1996); and Riley, D. P., et al., “Rational Design of Synthetic Enzymes and Their Potential Utility as Human Pharmaceuticals” (1997) in CatTech, I, 41. These mimics of superoxide dismutase have been shown to have a variety of therapeutic effects, including anti-inflammatory activity. See Weiss, R.H., et al., “Therapeutic Aspects of Manganese (II)-Based Superoxide Dismutase Mimics” In “Inorganic Chemistry in Medicine”, (Farrell, N., Ed.), Royal Society of Chemistry, in Press; Weiss, R. H., et al., “Manganese-Based Superoxide Dismutase Mimics: Design, Discovery and Pharmacologic Efficacies” (1995), In “The Oxygen Paradox” (Davies, K. J. A., and Ursini, F., Eds.) pp. 641-651, CLEUP University Press, Padova, Italy; Weiss, R. H., et al., J. Biol. Chem., 271: 26149 (1996); and Hardy, M. M., et al., J. Biol. Chem. 269: 18535-18540 (1994). Other non-proteinaceous catalysts which have been shown to have superoxide dismutating activity are the salen-transition metal cation complexes, as described in U.S. Pat. No. 5,696,109 and complexes of porphyrins with iron and manganese cations.

Current treatment strategies for ADC employ the use of HIV anti-viral agents, such as AZT, to limit the spread of the virus as opposed to treatment regimes tailored to block the triggering mechanism of apoptosis . This approach, however, is inherently limited because while it inhibits the spread of the virus, it does not prevent the apoptosis of brain cells associated with HIV infection. Treatment of ADC is further complicated because the compound administered must be able to cross the blood brain barrier in order to gain entry to the cerebral cavity and effectively inhibit the virus. For example, it is estimated that only about 50% of AZT taken orally, penetrates the brain. To further add to the difficulties in treating ADC, many compounds that do possess antioxidant activity in vivo, that may be anti-apoptotic, are non-selective and are also unable to cross the blood brain barrier.

Thus, a need exists for a treatment method wherein the compound administered is able to penetrate the blood brain barrier at a high rate. Furthermore, a need also exists for a compound able to prevent apoptosis of CNS cells upon its arrival in the cerebral cavity.

### SUMMARY OF THE INVENTION

Among the several aspects of the invention therefore is provided a method of preventing and/or treating HIV-mediated Central Nervous System damage, the method comprising administering to a subject a therapeutically effective amount of a composition comprising a non-proteinaceous catalyst for the dismutation of superoxide anions.

Another aspect provides a co-therapy for preventing and/or treating HIV-mediated Central Nervous System damage, the co-therapy comprising administering to a subject a therapeutically effective amount of a composition comprising non-proteinaceous a catalyst for the dismutation of superoxide anions and administering a therapeutically, prophylactically, pathologically, or resuscitative effective amount of a composition comprising an anti-viral drug.

Additional aspects provide a pharmaceutical composition for preventing and/or treating HIV-mediated central nervous system damage in a subject in need thereof, the composition comprising a therapeutically effective amount of a non-proteinaceous catalyst for the dismutation of superoxide anions, and a pharmaceutically acceptable carrier.

Yet another aspect provides a pharmaceutical composition for preventing and/or treating HIV-mediated central nervous system damage in a subject in need thereof, the composition comprising an effective amount of a non-proteinaceous catalyst for the dismutation of superoxide anions, an anti-viral drug and a pharmaceutically acceptable carrier.

A further aspect provides a method of inhibiting apoptotic neural cell death and/or apoptotic non-neural cell death, the method comprising administering to a subject a therapeutically effective amount of a composition comprising a non-proteinaceous catalyst for the dismutation of superoxide anions.

Still yet another aspect provides a method of inhibiting oxidative stress of neural cell death and/or apoptotic non-neural cell death, the method comprising administering to a subject a therapeutically effective amount of a composition comprising a non-proteinaceous catalyst for the dismutation of superoxide anions.

Other features of the present invention will be in part apparent to those of ordinary skill in the art and in part pointed out in the detailed description provided below.

### BRIEF DESCRIPTION OF THE DRAWINGS

These and other features, aspects, and advantages of the present invention will become better understood with regard to the following description, appended claims, and accompanying figures where:

FIG. 1 depicts the incubation of astroglial cells with supernatant of HIV-infected macrophages (M/M+HIV).

FIG. 2 is a graphic depiction of the affect of supernatant of HIV-1 infected macrophages (m/m) on the production of apoptotic cell death of astroglial cells as evaluated by FACS analysis. M40401 (10-100 μM), but not, L-NAME (100 μM) and AZT (50 μM) attenuated this effect. N=5 for each compound or control group.

FIG. 3 depicts immunocytochemical studies on HIV-related apoptosis. In a)—Incubation of astroglial cells with HIV-infected M/M leads to DNA fragmentation as shown by appearence, in immunocytochemical preparation, of TUNEL positive cells. In b) M40401 (100 μM), antagonized the generation of TUNEL positive astrocytes subsequent to incubation with HIV-infected M/M.

FIG. 4 depicts malondialdehyde (MDA; nmol g-1) increases within astroglial cells incubated with supernatants of HIV-infected macrophages (M/M +HIV) but not of Mock-infected cells (M/M+Mock). M40401 (100 μM) antagonized MDA overproduction, while L-NAME (100 μM) and AZT (50 μM) failed to antagonize lipid peroxydation.

FIG. 5 depicts additional immunocytochemical studies on HIV-related apoptosis. In a: a control cell line is shown: the cells are large with irregular nuclei composed, mainly, by euchromatin with a few peripheric heterochromatin: In the cytoplasm, are shown numerous dense mitochondria, dilated endoplasmic reticulum and cytoscheleton filaments. (×4900 magnification) In b: Incubation of astroglial cells with supernatant of not infected macrophages did not modify ultrastructural images of astroglial cells (×4900 magnification) In c, d, e,: After exposure for 3 h to supernatant of HIV-infected macrophages, astroglial cells undergo apoptotic cell death. In fact, the cells show an increase of plasma membrane protrusions and in many cells can be observed a developed cytoplasmic blebbing, large vacuoles due to cytoplasmic loss and cells in which cytoplasm is almost completely absent. The chromatin is condensed and marginalised, expressing DNA fragmentation. (×1900, ×2750 and ×3800 magnification). In f: The effect of HIV-infected macrophages on astroglial cells is strongly antagonised by co-incubation with M40401(100 μM). In particular, it is shown that cells maintain the normal architecture and the normal ratio between cytoplasm and nuclei which appear almost completely normal. In g and h : The coincubation with AZT (50 μM) an antiviral compound acting on HIV replication, failed to inhibit the pro-apoptotic effect of supernatant of HIV-infected macrophages, thus confirming that apoptosis is not due to the direct infection of astroglial cells by HIV.

### ABBREVIATIONS AND DEFINITIONS

To facilitate understanding of the invention, a number of terms and abbreviations as used herein are defined below:

The term “precursor ligand” means the organic ligand of a SOD mimic without the chelated transition metal cation and charge neutralizing anions.

The term “therapeutically effective amounts” means those amounts that, when administered to a particular subject in view of the nature and severity of that subject's disease or condition, will have the desired therapeutic effect, e.g., an amount which will cure, or at least partially arrest or prevent the disease or condition.

The term “substituted” means that the described moiety has one or more substituents comprising at least 1 carbon or heteroatom, and further comprising 0 to 22 carbon atoms, more preferably from 1 to 15 carbon atoms, and comprising 0 to 22, more preferably from 0 to 15, heteroatoms selected from the group consisting of: O, S, N, P, Si, B, F, Cl, Br, or I. These atoms may be arranged in a number of configurations, creating substituent groups which are unsaturated, saturated, or aromatic. Examples of such substituents include branched or unbranched alkyl, alkenyl, or alkynyl, cyclic, heterocyclic, aryl, heteroaryl, allyl, polycycloalkyl, polycycloaryl, polycycloheteroaryl, imines, aminoalkyl, hydroxyalkyl, hydroxyl, phenol, amine oxides, thioalkyl, carboalkoxyalkyl, carboxylic acids and their derivatives, keto, ether, aldehyde, amine, amide, nitrile, halo, thiol, sulfoxide, sulfone, sulfonic acid, sulfide, disulfide, phosphonic acid, phosphinic acid, acrylic acid, sulphonamides, amino acids, peptides, proteins, carbohydrates, nucleic acids, fatty acids, lipids, nitro, hydroxylamines; hydroxamic acids, thiocarbonyls, thiocarbonyls, borates, boranes, boraza, silyl, silaza, siloxy, and combinations thereof.

The term “alkyl”, alone or in combination, means a straight-chain or branched-chain alkyl radical containing from 1 to about 22 carbon atoms, preferably from about 1 to about 18 carbon atoms, and most preferably from about 1 to about 12 carbon atoms. Examples of such radicals include, but are not limited to, methyl, ethyl, n-propyl, isopropyl, n-butyl, isobutyl, sec-butyl, tert-butyl, pentyl, iso-amyl, hexyl, octyl, nonyl, decyl, dodecyl, tetradecyl, hexadecyl, octadecyl and eicosyl.

The term “alkenyl”, alone or in combination, means an alkyl radical having one or more double bonds. Examples of such alkenyl radicals include, but are not limited to, ethenyl, propenyl, 1-butenyl, cis-2-butenyl, trans-2-butenyl, iso-butylenyl, cis-2-pentenyl, trans-2-pentenyl, 3-methyl-1-butenyl, 2,3-dimethyl-2-butenyl, 1-pentenyl, 1-hexenyl, 1-octenyl, decenyl, dodecenyl, tetradecenyl, hexadecenyl, cis- and trans-9-octadecenyl, 1,3-pentadienyl, 2,4-pentadienyl, 2,3-pentadienyl, 1,3-hexadienyl, 2,4-hexadienyl, 5,8,11,14-eicosatetraenyl, and 9,12,15-octadecatrienyl.

The term “alkynyl”, alone or in combination, means an alkyl radical having one or more triple bonds. Examples of such alkynyl groups include, but are not limited to, ethynyl, propynyl (propargyl), 1-butynyl, 1-octynyl, 9-octadecynyl, 1,3-pentadiynyl, 2,4-pentadiynyl, 1,3-hexadiynyl, and 2,4-hexadiynyl.

The term “cycloalkyl”, alone or in combination means a cycloalkyl radical containing from 3 to about 10, preferably from 3 to about 8, and most preferably from 3 to about 6, carbon atoms. Examples of such cycloalkyl radicals include, but are not limited to, cyclopropyl, cyclobutyl, cyclopentyl, cyclohexyl, cycloheptyl, cyclooctyl, and perhydronaphthyl.

The term “cycloalkylalkyl” means an alkyl radical as defined above which is substituted by a cycloalkyl radical as defined above. Examples of cycloalkylalkyl radicals include, but are not limited to, cyclohexylmethyl, cyclopentylmethyl, (4-isopropylcyclohexyl)methyl, (4-t-butyl-cyclohexyl)methyl, 3-cyclohexylpropyl, 2-cyclohexylmethylpentyl, 3-cyclopentylmethylhexyl, 1-(4-neopentylcyclohexyl)methylhexyl, and 1-(4-isopropylcyclohexyl)methylheptyl.

The term “cycloalkylcycloalkyl” means a cycloalkyl radical as defined above which is substituted by another cycloalkyl radical as defined above. Examples of cycloalkylcycloalkyl radicals include, but are not limited to, cyclohexylcyclopentyl and cyclohexylcyclohexyl.

The term “cycloalkenyl”, alone or in combination, means a cycloalkyl radical having one or more double bonds. Examples of cycloalkenyl radicals include, but are not limited to, cyclopentenyl, cyclohexenyl, cyclooctenyl, cyclopentadienyl, cyclohexadienyl and cyclooctadienyl.

The term “cycloalkenylalkyl”, means an alkyl radical as defined above which is substituted by a cycloalkenyl radical as defined above. Examples of cycloalkenylalkyl radicals include, but are not limited to, 2-cyclohexen-1-ylmethyl, 1-cyclopenten-1-ylmethyl, 2-(1-cyclohexen-1-yl)ethyl, 3-(1-cyclopenten-1-yl)propyl, 1-(1-cyclohexen-1-ylmethyl)pentyl, 1-(1-cyclopenten-1-yl)hexyl, 6-(1-cyclohexen-1-yl)hexyl, 1-(1-cyclopenten-1-yl)nonyl and 1-(1-cyclohexen-1-yl)nonyl.

The terms “alkylcycloalkyl”, and “alkenylcycloalkyl”, mean a cycloalkyl radical as defined above which is substituted by an alkyl or alkenyl radical as defined above. Examples of alkylcycloalkyl and alkenylcycloalkyl radicals include, but are not limited to, 2-ethylcyclobutyl, 1-methylcyclopentyl, 1-hexylcyclopentyl, 1-methylcyclohexyl, 1-(9-octadecenyl)cyclopentyl and 1-(9-octadecenyl)cyclohexyl.

The terms “alkylcycloalkenyl” and “alkenylcycloalkenyl” means a cycloalkenyl radical as defined above which is substituted by an alkyl or alkenyl radical as defined above. Examples of alkylcycloalkenyl and alkenylcycloalkenyl radicals include, but are not limited to, 1-methyl-2-cyclopentyl, 1-hexyl-2-cyclopentenyl, 1-ethyl-2-cyclohexenyl, 1-butyl-2-cyclohexenyl, 1-(9-octadecenyl)-2-cyclohexenyl and 1-(2-pentenyl)-2-cyclohexenyl.

The term “aryl”, alone or in combination, means a phenyl or naphthyl radical which optionally carries one or more substituents selected from alkyl, cycloalkyl, cycloalkenyl, aryl, heterocycle, alkoxyaryl, alkaryl, alkoxy, halogen, hydroxy, amine, cyano, nitro, alkylthio, phenoxy, ether, trifluoromethyl and the like, such as phenyl, p-tolyl, 4-methoxyphenyl, 4-(tert-butoxy)phenyl, 4-fluorophenyl, 4-chlorophenyl, 4-hydroxyphenyl, 1-naphthyl, 2-naphthyl, and the like.

The term “aralkyl”, alone or in combination, means an alkyl or cycloalkyl radical as defined above in which one hydrogen atom is replaced by an aryl radical as defined above, such as benzyl, 2-phenylethyl, and the like.

The term “heterocyclic” means ring structures containing at least one other kind of atom, in addition to carbon, in the ring. The most common of the other kinds of atoms include nitrogen, oxygen and sulfur. Examples of heterocyclics include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups.

The term “saturated, partially saturated or unsaturated cyclic” means fused ring structures in which 2 carbons of the ring are also part of the fifteen-membered macrocyclic ligand. The ring structure can contain 3 to 20 carbon atoms, preferably 5 to 10 carbon atoms, and can also contain one or more other kinds of atoms in addition to carbon. The most common of the other kinds of atoms include nitrogen, oxygen and sulfur. The ring structure can also contain more than one ring.

The term “saturated, partially saturated or unsaturated ring structure” means a ring structure in which one carbon of the ring is also part of the fifteen-membered macrocyclic ligand. The ring structure can contain 3 to 20, preferably 5 to 10, carbon atoms and can also contain nitrogen, oxygen and/or sulfur atoms.

The term “nitrogen containing heterocycle” means ring structures in which 2 carbons and a nitrogen of the ring are also part of the fifteen-membered macrocyclic ligand. The ring structure can contain 2 to 20, preferably 4 to 10, carbon atoms, can be substituted or unsubstituted, partially or fully unsaturated or saturated, and can also contain nitrogen, oxygen and/or sulfur atoms in the portion of the ring which is not also part of the fifteen-membered macrocyclic ligand.

The term “organic acid anion” refers to carboxylic acid anions having from about 1 to about 18 carbon atoms.

The term “halide” means chloride, floride, iodide, or bromide.

As used herein, “R” groups means all of the R groups attached to the carbon atoms of the macrocycle, i.e., R, R′, R1, R′1, R2, R′2, R3, R′3, R4, R′4, R5, R′5, R6, R′6, R7, R′7, R8, R′8, R9, R′9.

### DESCRIPTION OF THE PREFERRED EMBODIMENTS

Applicants have discovered that treatment of a subject with a non-proteinaceous catalyst for the dismutation of superoxide anions results in diminishment of CNS apoptotic cell death associated with HIV infection. In addition, applicants have discovered that such apoptotic cell death results in whole or in part, from oxidative stress mediated by overproduction of superoxide anions. It is thus believed, without being bound by any particular theory or mechanism, that treatment of a subject with a non-proteinaceous catalyst for the dismutation of superoxide anions results in diminishment of HIV-related CNS apoptotic cell death by preventing oxidative stress mediated by superoxide anions.

Accordingly, the present invention provides a method to treat and/or prevent HIV-mediated CNS damage by administering to a subject a therapeutically, prophylactically, pathologically, or resuscitatitve effective amount of a composition comprising a non-proteinaceous catalyst for the dismutation of superoxide anions. The composition can contain a non-proteinaceous catalyst for the dismutation of superoxide anions alone or in combination with a HIV anti-viral agent. Additionally, pharmaceutical compositions are also provided.

### SOD Compounds

Preferably, the compound employed in the method of the present invention will comprise a non-proteinaceous catalyst for the dismutation of superoxide anions (“SOD mimic”) as opposed to a native form of the SOD enzyme. As utilized herein, the term “SOD mimic” means a low-molecular-weight catalyst for the conversion of superoxide anions into hydrogen peroxide and molecular oxygen. These catalysts consist of an organic ligand having a pentaazacyclopentadecane portion and a chelated transition metal ion, preferably manganese or iron. The term may include catalysts containing short-chain polypeptides (under 15 amino acids), or macrocyclic structures derived from amino acids, as the organic ligand. The term explicitly excludes a SOD enzyme obtained from any natural sources. SOD mimics are generally preferred for use in the method of the present invention because of the limitations associated with native SOD therapies such as, solution instability, limited cellular accessibility due to their size, immunogenicity, bell-shaped dose response curves, short half-lives, costs of production, and proteolytic digestion (Salvemini et al., (1999) Science 286: 304-306). For example, the best known native SOD, CuZn, has a molecular weight of 33,000 kD. Contrastingly, SOD mimics have an approximate molecular weight of 500 to 600 kD. The compounds employed in the method of the invention must be able to efficiently cross the blood brain barrier to penetrate the cerebral cavity in order to cause the dismutation of superoxide anions. Therefore, the smaller size exhibited by the SOD mimics is particularly advantageous for the present invention because it facilitates effective passage of the compound through the blood brain barrier and into the cerebral cavity.

In a particularly preferred embodiment, the SOD mimics utilized in the present invention comprise an organic ligand chelated to a metal ion. Particularly preferred catalysts are pentaaza-macrocyclic ligand compounds, more specifically the manganese(II), manganese (III), iron(II) and iron(III) chelates of pentaazacyclopentadecane compounds. The pentaaza macrocyclic ligand complexes of Mn(II) are particularly advantageous for use in the present invention because, in addition to a low molecular weight, they are highly selective for the dismutation of super oxide anions and possess catalytic rates similar or faster than native SOD counterparts. An example of this class of SOD mimic, designated M40401, is set forth in the examples below. These pentaazacyclopentadecane compounds can be represented by the following formula:

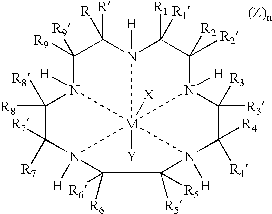

wherein M is a cation of a transition metal, preferably manganese or iron; wherein R, R′, R₁, R′₁, R₂, R′₂, R₃, R′₃, R₄ R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅ and R₆ or R′₆, R₇ or R′7 and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₂, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅ R′₅, R₆, R′₆, R₇, R′₇, R₈, R′₈, R₉ and R′₉ together with a different one of R, R₁, R₁, R′₁, R₂, R₃, R′₃, R₄, R′₄, R₅, R′₅, R₆, R′₆, R₇, R′₇, R₈, R′₈, R₉ and R′₉ which is attached to a different carbon atom in the macrocyclic ligand may be bound to form a strap represented by the formula:

(CH₂)ₓ—M—(CH2)_{w}—L—(CH2)—J—(CH2)_{y}—

wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof.

A preferred compound of this class of pentaazamacrocyclic class, designated M40401, is represented by the following formula:

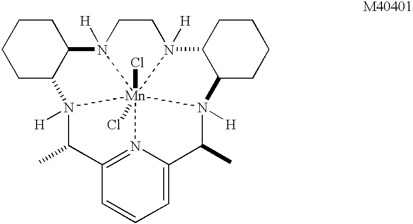

Yet another preferred compound of this class of pentaaza-macrocyclic class, designated, M40403, is represented by the following formula:

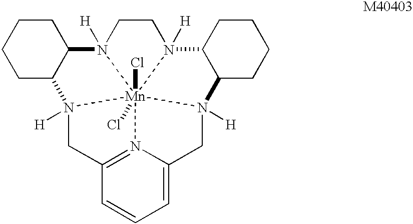

Still another preferred compound of this class of pentaaza-macrocyclic class, designated M40419, is represented by the following formula:

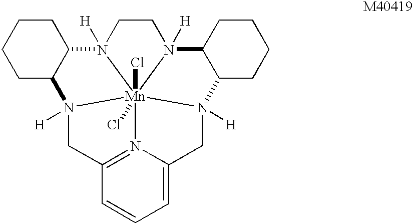

In another embodiment, the catalysts are substituted pentaaza-macrocyclic ligand compounds, which may be represented by the following formula:

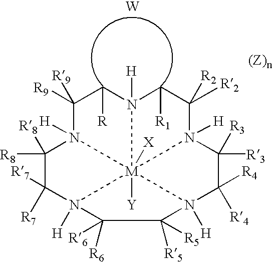

wherein a nitrogen of the macrocycle and the two adjacent carbon atoms to which it is attached independently form a substituted, unsaturated, nitrogen-containing heterocycle W having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and wherein R, R₁, R₂ R′₂, R₃, R′₃, R₄, R′₄, R₅, R 5, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; and, optionally, one or more of R₂ or R′₂ and R₃ or R′₃, R₄ or R′₄ and R₅ or R′₅, R₆ or R′₆ and R₇ or R′₇, or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and, optionally, one or more of R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and, optionally, one of R, R₁, R₂, R′₂, R₃, R′₃, R₄, R′₄, R₅, R 5, R₆, R′₆, R₇, R′₇, R₈, R′₈, R₉, and R′₉ together with a different one of R, R₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula

—(CH₂)ₓ—M—(CH₂)_{w}—L—(CH₂)_{z}—J—(CH₂)_{y}—

wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations of any of the above; wherein M is a cation of a transition metal selected from the group consisting of manganese and iron; and wherein X, Y and Z represent suitable ligands or charge-neutralizing anions which are derived from any monodentate or polydentate coordinating ligand or ligand system or the corresponding anion thereof(for example benzoic acid or benzoate anion, phenol or phenoxide anion, alcohol or alkoxide anion). X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins. The preferred ligands from which X, Y and Z are selected include halide, organic acid, nitrate and bicarbonate anions.

Particularly preferred substituted pentaaza-macrocyclic ligand compounds may be represented by the following formula:

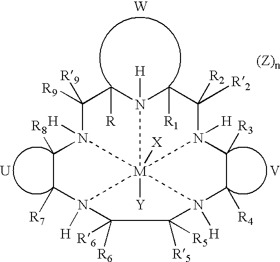

wherein the R groups, W, M, X, Y, and Z are as defined above, and wherein U and V are saturated cyclic structures, containing between 3 and 20, preferably between 4 and 10 carbon atoms and forming a cycloalkyl ring with the carbon atoms to which they are attached. In more preferred embodiments of the invention, U and V are two transcyclohexano fused rings. In particularly preferred embodiments of the invention, W is a substituted pyridine, and R, R₁, and the H on the nitrogen of the macrocycle within W are absent. Preferably, W is a substituted pyridine, and U and V are trans-cyclohexano fused rings. Preferred substituents on W are those which increase the potency of the catalyst for pharmaceutical applications. For instance, lipophilic substituents are preferred when the target of the catalyst is a hydrophobic tissue of the patient. In addition to altering the catalytic activity or log P and the concomitant targeting/pharmokinetic effects, applicants increase the potency of the catalyst for use in pharmaceutical compositions. These preferred substituents include cyclohexyl, hydroxyl alkyl thio, alkyl (2-thioacetic acid) esters, benzyloxy, methoxyarylthio, alkoxycarbonylarylthio, and aryl (2-thioacetic acid) esters.

The pentaaza-macrocyclic or substituted pentaaza-macrocyclic ligand compounds useful in the present invention can have any combinations of substituted or unsubstituted R groups, saturated, partially saturated or unsaturated cyclics, ring structures, nitrogen containing heterocycles, or straps as defined above. The “R” groups attached to the carbon atoms of the macrocycle can be in the axial or equatorial position relative to the macrocycle. When the “R” group is not hydrogen or when two adjacent “R” groups, i.e., on adjacent carbon atoms, together with the carbon atoms to which they are attached form a saturated, partially saturated or unsaturated cyclic or a nitrogen containing heterocycle, or when two R groups on the same carbon atom together with the carbon atom to which they are attached form a saturated, partially saturated or unsaturated ring structure, it is preferred that at least some of the “R” groups are in the equatorial position for reasons of improved activity and stability. This is particularly true when the complex contains more than one “R” group which is not hydrogen.

A wide variety of pentaaza-macrocyclic ligand compounds with superoxide dismutating activity may be readily synthesized. Generally, the transition metal center of the catalyst is thought to be the active site of catalysis, wherein the manganese or iron ion cycles between the (II) and (III) states. Thus, as long as the redox potential of the ion is in a range in which superoxide anion can reduce the oxidized metal and protonated superoxide can oxidize the reduced metal, and steric hindrance of the approach of the superoxide anion is minimal, the catalyst will function with a kcat of about 10⁻⁶ to 10⁻⁸.

The pentaaza-macrocyclic ligand compound catalysts described have been further described in U.S. Pat. No. 5,637,578, PCT application W098/58636, and copending application U.S. Ser. No. 09/398,120, all of which are hereby incorporated by reference. These pentaaza-macrocyclic ligand catalysts may be produced by the methods disclosed in U.S. Pat. No. 5,610,293. However, it is preferred that the pentaaza-macrocyclic ligand compound catalysts used in the present invention be synthesized by the template method described in copending applications U.S. Ser. No. 60/136,298 and U.S. Ser. No. 09/398,120, incorporated herein by reference.

Also suitable non-proteinaceous catalysts for use in the present invention, but less preferred than the pentaaza-macrocyclic ligand compounds, are the salen complexes of manganese and iron disclosed in U.S. Pat. No. 5,696,109, herein incorporated by reference. The term salen complex means a ligand complex with the general formula:

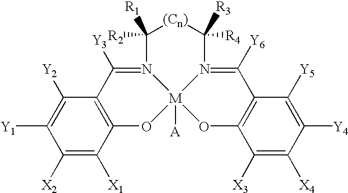

wherein M is a transition metal ion, preferably manganese or iron; A is an anion, typically Cl; and n is either 0, 1, or 2. X₁, X₂, X₃ and X₄ are independently selected from the group consisting of hydrogen, silyls, aryls, arylalkyls, primary alkyls, secondary alkyls, tertiary alkyls, alkoxys, aryloxys, aminos, quaternary amines, heteroatoms, and hydrogen; typically X₁ and X₃ are from the same functional group, usually hydrogen, quaternary amine, or tertiary butyl, and X₂ and X₄ are typically hydrogen. Y₁, Y_{2, Y}₃, Y₄, Y₅ and Y6 are independently selected from the group consisting of hydrogen, halides, alkyls, aryls, arylalkyls, silyl groups, aminos, alkyls or aryls bearing heteroatoms; aryloxys, alkoxys, and halide; preferably, Y₁ and Y₄ are alkoxy, halide, or amino groups. Typically, Y₁ and Y₄ are the same. R₁, R₂, R₃ and R₄ are independently selected from the group consisting of H, CH₃, C₂H₅, C₆H₅, O-benzyl, primary alkyls, fatty acid esters, substituted alkoxyaryls, heteroatom-bearing aromatic groups, arylalkyls, secondary alkyls, and tertiary alkyls. Methods of synthesizing these salen complexes are also disclosed in U.S. Pat. No. 5,696,109.

Iron or manganese porphyrins are also suitable non-proteinaceous catalysts for use in the present invention, such as, for example, Mn^{III}, tetrakis(4-N-methylpyridyl)porphyrin, Mn^{III} tetrakis-o-(4-N-methylisonicotinamidophenyl)porphyrin, Mn^{III} tetrakis(4-N-N-N-trimethylanilinium)porphyrin, Mn^{III}. tetrakis(1-methyl-4-pyridyl)porphyrin, Mn^{III} tetrakis(4-benzoic acid)porphyrin, Mn^{III} octabromo-meso-tetrakis(N-methylpyridinium-4-yl)porphyrin, 5, 10, 15, 20-tetrakis (2,4,6-trimethyl-3,5-disulfonatophenyl)-porphyrinato iron (III) (FeTMPS), Fe^{III} tetrakis(4-N-methylpyridyl)porphyrin, and Fe^{III} tetrakis-o-(4-N-methylisonicotinamidophenyl)porphyrin and preferably, substituted iron porphyin 5,10,15,20-tetrakis (2,4,6-trimethyl-3,5-disulfonatophenyl)-porphyrinato iron (III) (FeTMPS) may also be used in the methods and compositions of the present invention. See U.S. Patent No. 6,103,714. The catalytic activities and methods of purifying or synthesizing these non-proteinaceous catalysts are well known in the organic chemistry arts.

Activity of the compounds or complexes of the present invention for catalyzing the dismutation of superoxide can be demonstrated using the stopped-flow kinetic analysis technique as described in Riley, D. P. et al., Anal. Biochem., 196: 344-349 (1991) which is incorporated herein by reference. Stopped-flow kinetic analysis is an accurate and direct method for quantitatively monitoring the decay rates of superoxide in water. The stopped-flow kinetic analysis is suitable for screening compounds for SOD activity and activity of the compounds or complexes of the present invention, as shown by stopped-flow analysis, correlate to treating the above disease states and disorders.

Contemplated equivalents of the general formulas set forth above for the compounds and derivatives as well as the intermediates are compounds otherwise corresponding thereto and having the same general properties such as tautomers of the compounds and such as wherein one or more of the various R groups are simple variations of the substituents as defined therein, e.g., wherein R is a higher alkyl group than that indicated, or where the tosyl groups are other nitrogen or oxygen protecting groups or wherein the O-tosyl is a halide. Anions having a charge other than 1, e.g., carbonate, phosphate, and hydrogen phosphate, can be used instead of anions having a charge of 1, so long as they do not adversely affect the overall activity of the complex. However, using anions having a charge other than 1 will result in a slight modification of the general formula for the complex set forth above. In addition, where a substituent is designated as, or can be, a hydrogen, the exact chemical nature of a substituent which is other than hydrogen at that position, e.g., a hydrocarbyl radical or a halogen, hydroxy, amino and the like functional group, is not critical so long as it does not adversely affect the overall activity and/or synthesis procedure. Further, it is contemplated that manganese(III) complexes will be equivalent to the subject manganese(II) complexes.

### Co-Therapy

In a preferred embodiment, catalysts for the dismutation of superoxide are coupled with anti-viral agents to be used in the methods and compositions of the invention. Preferably, the anti-viral agent is AZT, ddI, ddC, KNI-272, dextran sulfate and any combination thereof. However, any anti-viral agent known in the art to be effective against HIV is within the scope of the present invention. Without being bound to any particular theory, it is believed that administration of a composition comprising a catalyst for dismutation of superoxide and an anti-viral agent to a subject infected with HIV will diminish HIV-mediated CNS damage by both inhibiting the spread of the virus and limiting apoptotic cell death mediated by superoxide anions. Thus, the coupling of catalysts for the dismutation of superoxide and anti-viral agents provides a synergistic therapy for the treatment of AIDS.

### Pharmaceutical Compositions

For use in treatment or prophylaxis of subjects, the compounds of the invention can be formulated as pharmaceutical or veterinary compositions. Depending on the subject to be treated, the mode of administration, and the type of treatment desired (e.g., inhibition, prevention, prophylaxis, therapy), the compounds are formulated in ways consonant with these parameters. The compositions of the present invention comprise a therapeutically or prophylactically effective dosage of a non-proteinaceous catalyst for the dismutation of superoxide. The catalyst for the dismutation of superoxide is preferably a SOD mimetic, as described in more detail above.

In another embodiment of the invention, pharmaceutical or veterinary compositions are provided which comprise non-proteinaceous catalysts for the dismutation of superoxide and anti-viral agents. The catalyst and anti-viral agent used in preparation of the pharmaceutical composition may be any such non-proteinaceous catalyst or anti-viral agent set-forth above.

When administered to a subject infected with HIV, these pharmaceutical compositions prevent HIV-related CNS damage, it is believed, by limiting apoptotic neural and non-neural triggered cell death mediated in whole or in part by superoxide anions.

The compositions of the present invention may be incorporated in conventional pharmaceutical formulations (e.g. injectable solutions) for use in treating humans or animals in need thereof. Pharmaceutical compositions can be administered by subcutaneous, intravenous, or intramuscular injection, or as large volume parenteral solutions and the like. The term parenteral as used herein includes subcutaneous injections, intravenous, intramuscular, intrasternal injection, or infusion techniques. For example, a parenteral therapeutic composition may comprise a sterile isotonic saline solution containing between 0.1 percent and 90 percent weight to volume of the catalysts for the dismutation of superoxide. A preferred solution contains from about 5 percent to about 20 percent, more preferably from about 5 percent to about 17 percent, more preferably from about 8 to about 14 percent, and most preferably about 10 percent catalysts for dismutation of superoxide in solution (% weight per volume).

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may e employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols which are solid at room temperature but liquid at the rectal temperature and will therefore melt in the rectum and release the drug.

Solid dosage forms for oral administration may include capsules, tablets, pills, powders, granules and gels. In such solid dosage forms, the active compound may be admixed with at least one inert diluent such as sucrose lactose or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

For administration to animal or human subjects, a typical dose of the composition comprising a catalyst for the dismutation of superoxide and an anti-viral agent may be readily determined by one skilled in the art employing any generally known method. Additionally, one skilled in the art will recognize that the total dosage will vary depending on the particular composition comprising a catalyst for the dismutation of superoxide and the anti-viral agent being administered.

The amount of active ingredient that may be combined with the carrier materials to produce a single dosage form will vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regimen for treating a disease condition with the compounds and/or compositions of this invention is selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely from subject to subject.

The pharmaceutical compositions of the present invention are preferably administered to a human. However, besides being useful for human treatment, these extracts are also useful for veterinary treatment of companion animals, exotic animals and farm animals, including mammals, rodents, avians, and the like. More preferred animals include horses, dogs, cats, sheep, and pigs.

### CNS Damage

As set forth above, the methods of the invention provide an effective means to treat HIV-related CNS damage. CNS damage, as utilized herein, shall predominantly include ADC. However, the methods of the invention also are effective in treating and/or preventing other CNS damage mediated by HIV.

The detailed description set-forth above is provided to aid those skilled in the art in practicing the present invention. Even so, this detailed description should not be construed to unduly limit the present invention as modifications and variation in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

The ability of SODm to attenuate the effect of HIV-mediated CNS damage was evaluated. In particular, the present experiments investigate the role of superoxide anions in the apoptotic cell death of astroglial cells incubated with supernatants of HIV-infected macrophages and demonstrate the protective effect of the SODm, designated M40401 and related compounds on HIV-related apoptosis of astroglial cells. M40401 is a preferred compound of the pentaaza-macrocyclic class of SODm and is represented by the following formula:

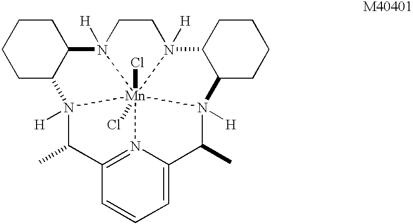

Materials and Methods

Cell Cultures

Human Primary Macrophages.

Peripheral blood mononuclear cells (PBMCs) were obtained from the blood of healthy seronegative donors by separation over Ficoll-Hypaque gradient. After separation, PBMCs were seeded at a density of 6×10⁶ cells/ ml in 25 cm² plastic flasks in RPMI 1640 with the addition of 50 units/ml penicillin, 50 ug/ml streptomycin, 2 mM L-glutamine, and 20% heat-inactivated, mycoplasma- and endotoxin-free FCS. Cells were incubated at 37° C. in humidified air containing 5% CO₂. After 5 days of culture, non-adherent cells were removed by repeated washing with warm medium. Macrophages obtained with this method resulted in >95% of purity by cytofluorimetric analysis.

Human Astrocytoma Cell Line.

The astrocytic cell line Lipari was derived from a 51 year old male patient who presented a large right front-temporal mass (astrocytoma), and grown as previously described (Mollace, V., Colasanti, M., Rodinò, P., Lauro, G. M. & Nisticò, G. (1994b). Biochem. Biophys. Res. Commun. 203, 87-92.). Cells were expanded and cultured by seeding them in 25cm plastic flasks at a density of 0.7×10⁶ cells/flask in complete medium, and incubated at 37° C. in humidified air containing 5% CO₂.

Infection of Human Macrophages by HIV-1.

A monocytotropic strain of HIV-1, named HIV-1_{Ba-L} was used in all experiments. M/M cultures were exposed to HIV and virus expansion was performed. After 2 hours, virus was removed by washing cultures with warm medium and then cultures of M/M infected by HIV were carried out for further 13-15 days.

Challenge of Astrocytes.

The supernatant of HIV-infected M/M was collected 13 days after virus infection and then incubated for 3 hours with cultured astrocytes. The supernatant of Mock-infected M/M was used as control treatment. When required M40401 (10-100μM), L-NAME (100μM) and AZT (50 μM) were added immediately before exposure to supernatants of HIV-infected or Mock-infected M/M. Cells were then carefully and repeatedly washed and cultured in complete medium with or without treatment. HIV-p24 antigen production in supernatants of M/M was assesed using a commercially available ELISA Kit (HIV-p24 gag Abbott Lab, Pomezia, Italy).

Trypan Blue Exclusion Test of Cell Viability.

The dye exclusion test was used to determine the number of viable cells after exposure of astrocytes treated or not treated with supernatants. At 8 days after treatment, astrocytes were trypsinized, exposed to dye, and then visually examined to determine whether cells take up or exclude dye. The live cells that posses intact cell membranes exclude trypan blue, whereas dead cells do not.

Evaluation of Programmed Cell Death in Cultured Astrocytes Exposed to Supernatant of HIV-Infected Macrophages.

FACS analysis.

The astrocytic cells either treated or untreated with supernatants of HIV-infected macrophages were gently detached from plastic 8 days after exposure. Aliquots of 5×10⁵ cells were centrifuged at 300 g for 5 min; pellets were washed with PBS, placed on ice, and overlaid with 0.5 ml of a hypotonic fluorocrome solution containing 50 ug/ml propidium iodide, 0.1% sodium citrate, and 0.1% Triton X-100. After gentle resuspension in this solution, cells were left at 4° C. for 30 min, in absence of light, before analysis. Propidium iodide-stained cells were analysed with a FACScan Flow Cytometer; fluorescence was measured between 565 and 605nm. The data were acquired and analysed by the Lysis II program.

Immunocytochemical Studies.

Astrocytic apoptotic nuclei were assessed by in situ terminal deoxynucleotidyl transferase (TdT)-mediated dUTP-biotin nick-end labeling (TUNEL) of DNA strand breaks. Briefly, astrocytic cells were permeabilized by a 20 min incubation at room temperature in 0.15 Triton X/0.15 sodium citrate, washed in 10 mM PBS pH 7.2, reacted for 1 hour at 37° C. with TdT and biotin-labeled dUTP in 30 mM Tris-HCl, pH 7.2, 140 mM sodium cacodylate, 1 mM cobalt chloride, and visualized using streptavidin-alcalin phosphatase complex with natol-fast red. Cells were coverslipped under DPX mounting and the number of TUNEL-positive cells were counted. Negative controls included sections incubated with biotin-labeled dUTP in the absence of TdT.

Ultrastructural Studies.

Cells for electron microscopy were fixed in 2.5% glutaraldehyde in PBS pH7.4 at 4° C. and then washed for 2 hours in PBS and post fixed in osmium tetroxide 1,33% for 2 hours at 4° C. After several washes in PBS, the cells were dehydrated in graded alcohol, transferred into toluene, and embedded in Epon 812 resin. The resin was allowed to polymerize in a dry oven at 60° C. for 24 hours. Thin sections were cut with a glass knife Reichert microtome, stained with Touloidine Blue and examined on Axioscope microscope. Ultra-thin sections were cut on a Reichert microtome using a diamond knife, stained with uranyl-acetate-lead-hydroxide and evaluated and photographed on a Philips electron microscope CM 10 (Philips).

Selectivity of M40401:

In a series of papers (Dawson et al., (1993) Proc Nat Acad USA 90(8):3256-9 ), we have shown that the pentaaza macrocyclic ligand complexes of Mn(II) can not only be highly active catalysts for the dismutation of O₂⁻, but that they are also highly selective. The pentaaza macrocyclic compound designated SC-55858 (Dawson et al., (1993) Proc Nat Acad USA 90(8):3256-9 ), for example, has been shown to catalytically dismute O₂^{-.} at a rate exceeding 10⁺⁸ molecules of O₂^{-.} per molecule of complex per second at pH=7.4 and 21° C., at a rate comparable to the native Mn SOD enzyme. Remarkably, this complex and others of this pentaaza macroclcic ligand class, such as M40403 or M40401, described in the references above, do not react with hydrogen peroxide under the same conditions (Dawson et al., (1993) Proc Nat Acad USA 90(8):3256-9; and Palamara et al., (1996). AIDS Res Hum Retroviruses. 12(16):1537-41 ), nor do they react with other biologically relevant oxidants such as ONOO— or nitric oxide. Thus, in our assays to assess catalytic catalase activity using oxygen electrodes (Premanathan et al, (1997) AIDS Res Hum Retroviruses 13(4):283-90; and Edeas et al., (1997) Free Radic Biol Med 23(4):571-8 ) in which total oxygen concentration evolved from the reaction of hydrogen peroxide with catalase (or any putative catalase mimic) is quantitatively monitored, no catalytic activity is observed between SC-55858, M40403, or M40401 and H₂O₂, and further no stoichiometric reaction is observed to occur between these complexes and H₂O₂ as monitored via spectrophotometric or electrochemical (cyclic voltammetric) techniques. The stopped flow assay developed for monitoring peroxynitrite (PN) catalytic activity ( Nottet et al., (1994) J Leukoc Biol 56(6):702-7 ) was also utilized to assess the PN activity of these agents. No catalytic or stoichiometric reactivity of PN with these complexes is observed. These substrate specificities allow us to probe directly the biological roles that the free radical, O₂^{-.}, plays by studying the effects that such selective catalysts exhibit in vivo.

Results

Incubation of human macrophages (M/M) for 3 h with HIV-1 produced a prominent and significant generation of p24 in the cell supernatant (30000±1250 pg/ml; n=4) 10-15 days after the incubation, thus indicating that HIV was able to interact and to infect M/M.

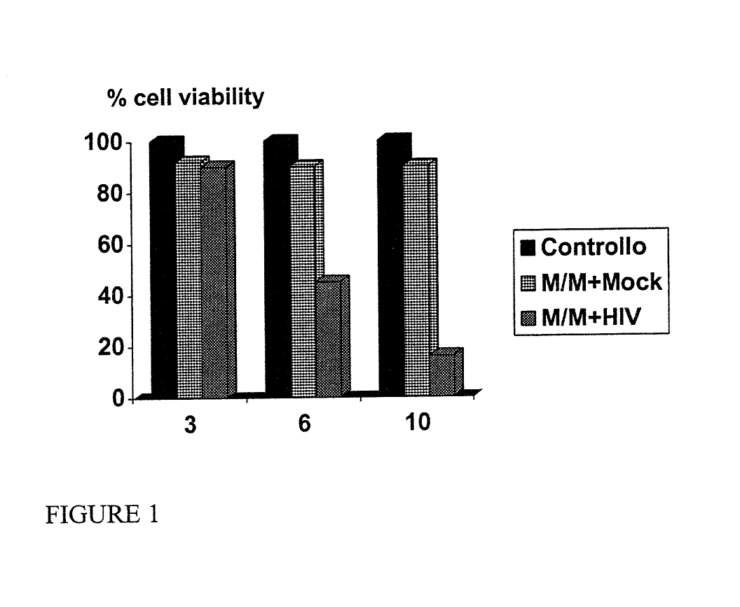

When supernatant of HIV-infected M/M was incubated for 2 h with Lipari astroglial cell line, a reduced viability of astrocytes was seen as evaluated by trypan blue exclusion method, reaching its maximum 6-10 days after the incubation with M/M supernatant was carried out (FIG. 1). Supernatant of control M/M or Mock-infected M/M did not alter astroglial cell viability.

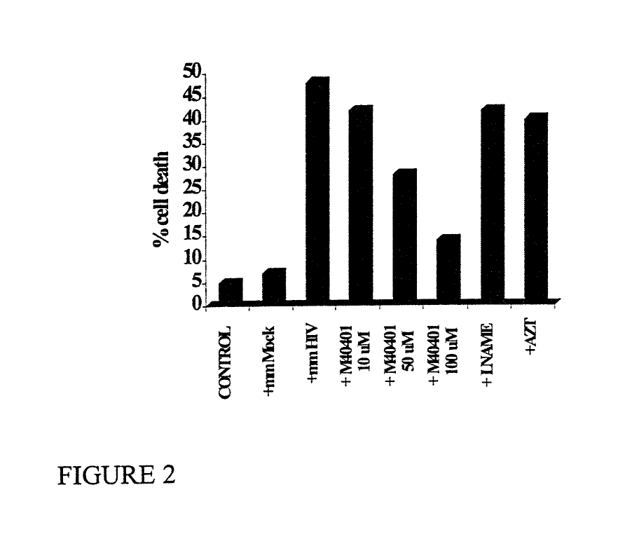

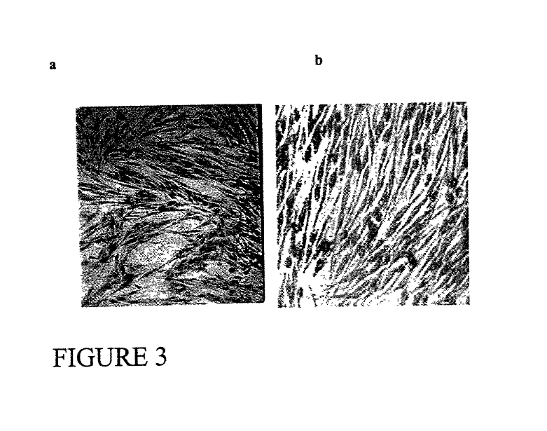

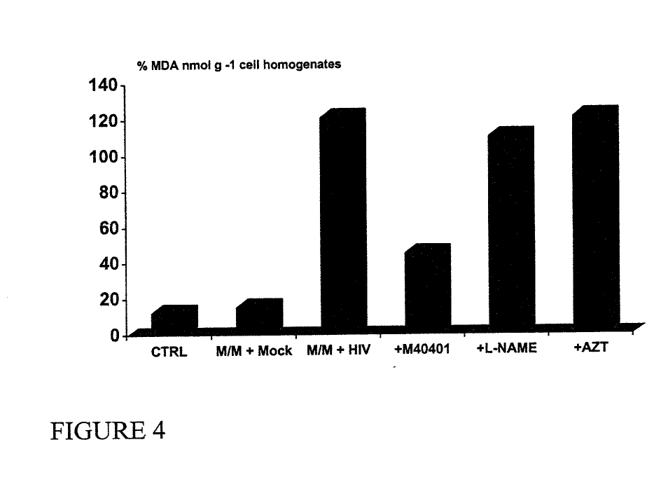

This effect was mainly related to apoptotic cell death of astroglial cells. Indeed, cytofluorimetric analysis of treated cells (FACS) showed a significant apoptotic cell death (FIG. 2), an effect which was confirmed by data coming from immunocytochemical analysis of DNA fragmentation by using TUNEL reaction (FIG. 3a and 3b). The apoptosis of astroglial cells related to their incubation with supernatants of HIV-infected M/M was accompanied by an increased generation of superoxide anions, as shown by the prominent rise in malondialdehyde (MDA) in the homogenates of astrocytes undergoing apoptotic cell death (FIG. 4). Neither apoptotic phenomena, nor MDA overproduction were generated after incubation of astroglial cells with control M/M or with Mock-infected M/M (FIG. 1, 2 and 4). Pretreatment of cells with M40401 (10-100 μM; n=4), significantly antagonized both MDA formation and apoptotic cell death produced in cultured astroglial cells by incubation with supernatants of HIV-1 infected M/M, as shown by FACS analysis and TUNEL reaction, while L-NAME (an NO synthase inhibitor; 100 μM; n=4) and AZT (an anti-viral compound acting on HIV-replicative cycle; 50 μM; n=4), failed to modify apoptotis of astroglial cells (FIG. 3).

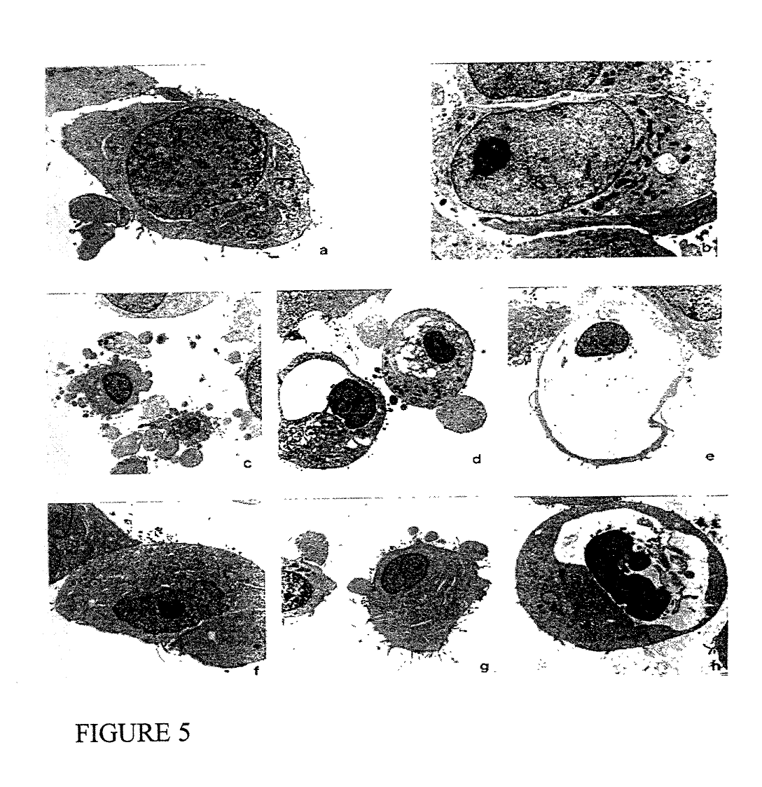

These results were confirmed by ultrastructural studies by means of electron microscopy. In particular, control astroglial cells as well as cells treated with supernatant of Mock-infected M/M, showed large size with irregular nuclei composed, mainly, by euchromatin with a few peripheric heterochromatin associated, in the cytoplasm, with numerous dense mitochondria, dilated endoplasmic reticulum and cytoscheleton filaments. (FIG. 5a and b). After exposure for 2 h to supernatant of HIV-infected M/M, astroglial cells underwent apoptotic cell death which became evident 6-10 days after exposure of cells to the supernatants of infected M/M. In fact, astrocytes showed an increase of plasma membrane protrusions and, in many cells, was seen a developed cytoplasmic blebbing, large vacuoles due to cytoplasmic loss and cells in which cytoplasm was almost completely absent. The chromatin was seen condensed and marginalized, expressing DNA fragmentation. (FIG. 5c, d and e). The effect of HIV-infected M/M on astroglial cells was strongly antagonized by co-incubation with M40401 (10-100 μM; n=4). In particular, we found that, in M40401-pretreated astrocytes, cells maintained the normal architecture and the normal ratio between cytoplasm and nuclei appeared almost completely normal (FIG. 5f). The co-incubation with the anti-viral AZT (50 μM; n=4) or with the NOS inhibitor L-NAME (100 μM; n=4), failed to inhibit the pro-apoptotic effect of supernatant of HIV-infected macrophages, thus confirming that apoptosis, in our system, was not due to the direct infection of astroglial cells by HIV or by abnormal activation of NO synthase in this system (FIG. 5g and h). M40401 did also not affect, when incubated in the absence of M/M supernatants, astroglial cell viability and ultrastructure (not shown).

The present experiments show, for the first time, that incubation of human cultured astroglial cells with supernatant of HIV-infected human macrophages leads to apoptotic cell death of astrocytes, an effect which is driven by overproduction of superoxide anions. This is expressed by sustained generation of thiobarbituric-reactive products (which shows the occurrence of lipid peroxidation) in astroglial cells undergoing HIV-related apoptosis, with both effects being antagonized by the non-peptidic SOD mimic, M40401, in a dose-dependent manner. This result is relevant since it shows, for the first time, that the antagonism of HIV-related oxidative stress by molecules such as novel SOD mimics able to cross the brain blood barrier, is crucial in reducing the main cause of cell death occurring within brain tissues in patients undergoing AIDS. In addition, our data highlight the role of superoxide anions (and possibly the subsequent activation of apoptotic program) as a final common pathway through which pro-inflammatory substances, released by infected macrophages/microglial cells, may produce neuronal cell death in neuroAIDS, an effect now clearly blocked by selective antioxidants such as M40401.

What is claims is:

1. A method of preventing and/or treating HIV-mediated Central Nervous System damage, the method comprising administering to a subject a therapeutically effective amount of a composition comprising a non-proteinaceous catalyst for the dismutation of superoxide anions.

2. The method of claim 1 wherein prevention and/or treatment of HIV-mediated Central Nervous System damage is achieved by inhibiting oxidative stress of neural cells and/or non-neural cells.

3. The method of claim 2 wherein the oxidative stress is mediated by superoxide anions.

4. The method of claim 1 wherein prevention or treatment of HIV-mediated Central Nervous System damage is achieved by limiting apoptotic neural cell death and/or apoptotic non-neural cell death.

5. The method of claim 4 wherein apoptotic cell death is limited by preventing oxidative stress mediated by superoxide anions.

6. The method of claim 1 wherein the Central Nervous System damage is AIDS-dementia complex.

7. The method of claim 1 wherein the catalyst comprises an organic ligand chelated to a metal ion selected from the group of manganese(II), manganese(III), iron(II) and iron(III).

8. The method of claim 7 wherein the catalyst is a pentaaza-macrocyclic ligand complex or a substituted pentaaza-macrocyclic ligand complex.

9. The method of claim 8 wherein the pentaazamacrocyclic ligand complex is represented by the following formula: 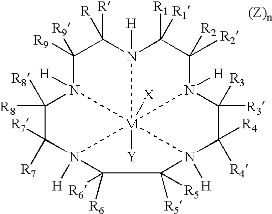
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁ R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂ R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

10. The method of claim 8 wherein the substituted pentaaza-macrocyclic ligand complex is represented by the following formula: 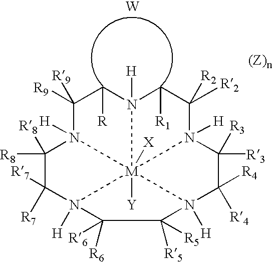
wherein a nitrogen of the macrocycle and the two adjacent carbon atoms to which it is attached independently form a substituted, unsaturated, nitrogen-containing heterocycle W having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and wherein R, R₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, cycloalkylcycloalkenyl, alkenylcycloalkyl, alkenylcycloalkenyl, heterocyclic, aryl and aralkyl radicals; and, optionally, one or more of R₂ or R′₂ and R₃ or R′₃, R₄ or R′₄ and R₅ or R′₅, R₆, or R′₆ and R₇ or R′₇, or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent; and, optionally, one or more of R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms;
and, optionally, one of R, R₁, R₂, R′₂, R₃, R′₃, R₄, R′₄, R₅, R′₅, R₆, R′₆, R₇, R′₇, R₈, R′₈, R₉, and R′₉ together with a different one of R, R₁,, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and
combinations of any of the above; wherein M is a cation of a transition metal selected from the group consisting of manganese and iron; and wherein X, Y and Z represent suitable ligands or charge-neutralizing anions which are derived from any monodentate or polydentate coordinating ligand or ligand system or the corresponding anion thereof.

11. The method of claim 10 wherein the substituted pentaaza-macrocyclic ligand is further described by the formula: 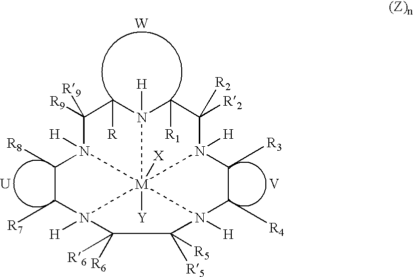
wherein U and V are saturated cyclic structures containing between 3 and 20 carbon atoms and form a cycloalkyl ring with the carbon atoms of the macrocycle to which they are attached.

12. The method of claim 11 wherein W is a substituted pyridino moiety.

13. The method of claim 11 wherein U and V are transcyclohexanyl fused rings and W is a substituted pyridino moiety.

14. The method of claim 7 wherein the catalyst is a porphyrin ligand complex or a substituted porphyrin ligand complex.

15. The method of claim 14 wherein the porphyrin ligand complex is selected from the group consisting of 175 manganese (II) porphyrin complexes, manganese(III) porphyrin complexes, iron (II) porphyrin complexes, and iron(III) porphyrin complexes.

16. The method of claim 15 wherein the porphyrin ligand complex is a 5,10,15,20-tetrakis (2,4,6-trimethyl-3,5-disulfonatophenyl)-porphyrinato iron (III) (FeTMPS).

17. The method of claim 1 wherein the subject is a mammal.

18. The method of claim 17 wherein the mammal is a human.

19. A co-therapy for preventing and/or treating HIV-mediated Central Nervous System damage, the co-therapy comprising administering to a subject a therapeutically effective amount of a composition comprising a non-proteinaceous catalyst for the dismutation of superoxide anions and administering a therapeutically effective amount of a composition comprising an anti-viral drug.

20. The method of claim 19 wherein the catalyst comprises an organic ligand chelated to a metal ion selected from the group of manganese(II), manganese(III), iron(II) and iron(III).

21. The method of claim 20 wherein the catalyst is a pentaaza-macrocyclic ligand complex or a substituted pentaaza-macrocyclic ligand complex.

22. The method of claim 21 wherein the pentaazamacrocyclic ligand complex is represented by the following formula: 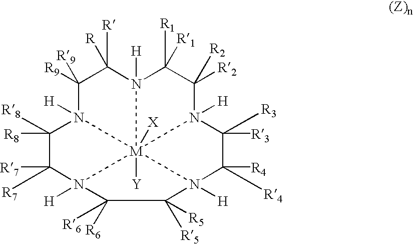
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′_{5,} R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂ R₃, R′₃, R₄, R′₄, R₅, R′₅, R₆, R′₆, R₇, R′₇, R₈, R′₈, R₉, and R′₉ together with a different one of R, R , R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

23. The method of claim 21 wherein the substituted pentaaza-macrocyclic ligand complex is represented by the following formula: 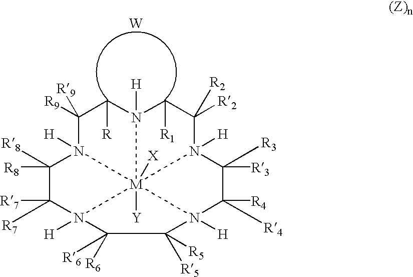
wherein a nitrogen of the macrocycle and the two adjacent carbon atoms to which it is attached independently form a substituted, unsaturated, nitrogen-containing heterocycle W having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent;
and wherein R, R₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals;
and, optionally, one or more of R₂ or R′₂ and R₃ or R′₃, R₄ or R′₄ and R₅ or R′₅, R₆ or R′₆ and R₇, or R′₇ or R₈ or R′₈ and R₉ or R′₉ together with the carbon atoms to which they are attached independently form a substituted or unsubstituted nitrogen containing heterocycle having 2 to 20 carbon atoms, which may be an aromatic heterocycle, in which case the hydrogen attached to the nitrogen which is both part of the heterocycle and the macrocycle and the R groups attached to the carbon atoms which are both part of the heterocycle and the macrocycle are absent;
and, optionally, one or more of R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms;
and, optionally, one of R, R₁, R₂, R₂, R₃, R′₃, R₄, R′₄, R₅, R′₅, R₆, R′₆, R₇, R′₇, R₈, R′₈, R₉, and R′₉ together with a different one of R, R₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ—M—(CH₂)_{w}—L—(CH₂)_{z}—J—(CH₂)_{y}—
wherein w, x, y and z independently are integers from 0 to 10 and M, L and J are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof;
and combinations of any of the above;
wherein M is a cation of a transition metal selected from the group consisting of manganese and iron;
and wherein X, Y and Z represent suitable ligands or charge-neutralizing anions which are derived from any monodentate or polydentate coordinating ligand or ligand system or the corresponding anion thereof.

24. The method of claim 23 wherein the substituted pentaaza-macrocyclic ligand complex is further described by the formula: 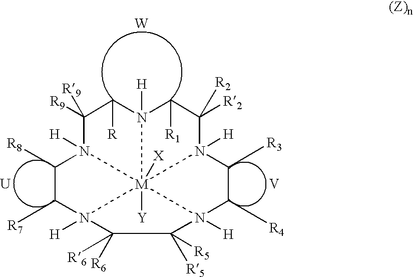
wherein U and V are saturated cyclic structures containing 160 between 3 and 20 carbon atoms and form a cycloalkyl ring with the carbon atoms of the macrocycle to which they are attached.

25. The method of claim 24 wherein W is a substituted pyridino moiety.

26. The method of claim 24 wherein U and V are transcyclohexanyl fused rings and W is a substituted pyridino moiety.

27. The method of claim 19 wherein the catalyst is a porphyrin ligand complex or a substituted porphyrin ligand 170 complex.

28. The method of claim 27 wherein the porphyrin ligand complex is selected from the group consisting of manganese (II) porphyrin complexes, manganese(III) porphyrin complexes, iron (II) porphyrin complexes, and iron(III) porphyrin complexes.

29. The method of claim 28 wherein the porphyrin ligand complex is a 5,10,15,20-tetrakis (2,4,6-trimethyl-3,5-disulfonatophenyl)-porphyrinato iron (III) (FeTMPS).

30. The method of claim 19 wherein the anti-viral drug is selected from the group consisting of AZT, ddI, ddC, KNI-272, and dextran sulfate.

31. The method of claim 19 wherein the catalyst and the anti-viral drug are administered in a substantially simultaneous manner.

32. The method of claim 19 wherein the catalyst and the anti-viral drug are administered in a sequential manner.

33. The method of claim 19 wherein the subject is a mammal.

34. The method of claim 33 wherein the mammal is a human.

35. A pharmaceutical composition for preventing and/or treating HIV-mediated central nervous system damage in a subject in need thereof, the composition comprising a therapeutically effective amount of a non-proteinaceous catalyst for the dismutation of superoxide anions, and a pharmaceutically acceptable carrier.

36. A pharmaceutical composition for preventing and/or treating HIV-mediated central nervous system damage in a subject in need thereof, the composition comprising an effective amount of a non-proteinaceous catalyst for the dismutation of superoxide anions, an anti-viral drug and a pharmaceutically acceptable carrier.

37. A method of inhibiting apoptotic neural cell death and/or apoptotic non-neural cell death, the method comprising administering to a subject a therapeutically effective amount of a composition comprising a non-proteinaceous catalyst for the dismutation of superoxide anions.

38. The method of claim 37 wherein the cells are selected from the group consisting of microglia cells, monocytes, macrophages, and astroglia cells.

39. A method of inhibiting oxidative stress of neural cell death and/or apoptotic non-neural cell death, the method comprising administering to a subject a therapeutically effective amount of a composition comprising a non-proteinaceous catalyst for the dismutation of superoxide anions.

40. The method of claim 39 wherein the cells are selected from the group consisting of microglia cells, monocytes, macrophages, and astroglia cells.

41. The method of claim 1 wherein the catalyst for the dismutation of superoxide anions is further described by the formula:

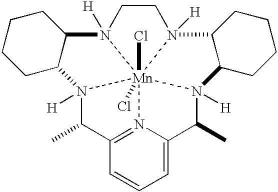

42. The method of claim 1 wherein the catalyst for the dismutation of superoxide anions is further described by the formula:

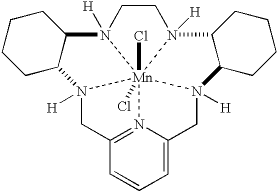

43. The method of claim 1 wherein the catalyst for the dismutation of superoxide anions is further described by the formula: [chemical structure]
